(19) United States
(12) Patent Application Publication
(10) Pub. No.: US 2022/0125438 A1
(43) Pub. Date: Apr. 28, 2022

(54) CARDIAC IMPLANTABLE ELECTRONIC DEVICE POCKET COMPRESSION APPARATUS AND METHOD OF MITIGATING LOCALIZED BLEEDING USING SAME

(71) Applicants: Dhanunjaya Lakkireddy, Leawood, KS (US); Srijoy Mahapatra, Edina, MN (US)

(72) Inventors: Dhanunjaya Lakkireddy, Leawood, KS (US); Srijoy Mahapatra, Edina, MN (US)

(21) Appl. No.: 17/571,247

(22) Filed: Jan. 7, 2022

Related U.S. Application Data
(63) Continuation-in-part of application No. 16/940,133, filed on Jul. 27, 2020.
Application No. 16/940,133 is a division of application No. 15/292,697, filed on Oct. 13, 2016, now Pat. No. 10,722,244.
Application No. 15/292,697 is a continuation of application No. 29/707,724, filed on Sep. 30, 2019.
(60) Provisional application No. 62/240,967, filed on Oct. 13, 2015.

(51) Int. Cl. A61B 17/132; A41D 13/12

(52) U.S. Cl. CPC A61B 17/1325; A61B 2017/12004; A41D 13/1245

(57) ABSTRACT

An apparatus and method for mitigating pocket bleeding and hematoma formation in a patient. A wrap having a block is secured around the torso of the patient to align the block with a torso area. The wrap includes a first portion extending laterally around the torso and a second portion extending over the shoulder and connecting to the first portion. The block is secured within a pouch of the wrap over the torso area. The first and second portions are adjustable to apply an adjustable force to the block that produces a corresponding adjustable pressure on the torso area. The applied pressure is high enough to hinder blood flow to the torso area to promote clotting at the torso area and low enough to permit blood flow to the torso area to promote healing.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This patent application is a continuation-in-part of U.S. patent application Ser. No. 16/940,133, filed Jul. 27, 2020, entitled “Cardiac Implantable Electronic Device Pocket Compression Apparatus and Method of Mitigating Localized Bleeding Using Same,” which is a divisional of U.S. patent application Ser. No. 15/292,697, filed Oct. 13, 2016, entitled, “Cardiac Implantable Electronic Device Pocket Compression Apparatus and Method of Mitigating Localized Bleeding Using Same,” which claims priority to U.S. provisional patent application No. 62/240,967, filed Oct. 13, 2015, entitled “Implantable Cardiac Device Pocket Compression Apparatus,” the disclosure of each of which is hereby incorporated by reference herein in its entirety for all purposes and forms a part of this specification.

This patent application is a continuation of U.S. design patent application Ser. No. 29/707724 filed Sep. 30, 2019, entitled “Compression Apparatus,” the disclosure of which is hereby incorporated by reference herein in its entirety for all purposes and forms a part of this specification.

### BACKGROUND

### Field of the Invention

The invention generally relates to a medical apparatus and, more particularly, the invention relates to a treatment apparatus for use in connection with cardiac device implantations.

### Description of the Related Art

Several hundred thousands of patients receive subcutaneous cardiac devices (pacemakers, defibrillators, resynchronization therapy devices, loop recorders) throughout the world each year, with many thousands in the United States alone. With increasing advances in medical devices and the advent of life saving implantable cardioverter defibrillators along with cardiac resynchronization therapy, cardiac device therapy placed in the chest through the infraclavicular/subclavian approach has become very common. Usually, after sterile preparation, a small incision is made below the clavicle either on the right or left side. A subcutaneous pocket is created, bleeders are secured, and access to the subclavian vein or cephalic vein is obtained under the guidance of fluoroscopy. Then pacing or defibrillator leads may be placed in the appropriate chambers of the heart. These leads may be secured to the fascia over the pectoralis muscle and the end of which may be usually plugged into the ports in the device generator. The portion of the leads and the generator may be placed in the pocket and closed in two or three layers of suture. Bleeders may be secured before closing the pocket. Sterile strips may be placed over the closed incision and allowed to heal over the next few days.

Based on prior research, pocket bleeding and hematoma formation may be seen in about 4.9% patients and the incidence may be higher in those receiving defibrillators compared to pacemakers. Pocket bleeding can prolong hospital stay in 1.5% of patients who receive these devices up to 2.5 days with about 0.5% requiring repeat hospitalization and close to 1% needing pocket revision and hematoma evacuation. The risk of post device bleeding and hematoma formation may be significantly higher (e.g., up to 12%-20%) in those patients who may be bridged with anticoagulants like heparin and enoxaparin. Even in those cases where warfarin is continued throughout the procedure, the risk of post procedural bleeding may be up to 2-6%. Often, large hematomas and subsequent evacuations lead to increased incidence of infection.

Commonly, small swelling or hematoma left untreated and healing are left to normal body processes. However, hematomas and pocket bleeding can be serious enough in a certain percentage of patients as described above, requiring immediate attention. This can significantly increase the cost of treatment, and the amount of lost wages. This can also add a significant morbidity to the patient's health. Patients with device site hematomas and bleeding are at higher risk for infections which often require hospitalizations, intravenous antibiotic therapy and device extraction that leads to significant procedural risks, costs and patient comfort

### SUMMARY

In accordance with one embodiment of the invention, a medical device for applying pressure to a localized area of patient's body includes a pliable wrap for wrapping around at least a portion of the patient, and a block of non-inflatable or inflatable material positioned between the wrap and the localized area of the patient. The wrap may be a vest for holding the block against the patient's torso, or a sleeve to hold the block against a patient's arm or a leg, to name but a few examples. In some embodiments, the wrap includes an elastic material, such as neoprene for example, and may also include straps to adjust the pressure applied to a localized area on the patient by the block.

The block is sized and shaped to apply pressure to the localized area of the patient, which pressure is sufficient to hinder but not completely prevent blood flow in the localized area of the patient's body. For example, the block may have a wedge shape with a narrow face positioned to face the patient when the medical device is worn by the patient, or an opening therethrough that surrounds the wound site.

Some embodiments include a sealable pouch in the medical device for securing the block in position, and some embodiments also include an absorbent sheath around the block within the pouch. Moreover, some embodiments include at least one filler member within the pouch to secure the location of the block within the pouch. Other embodiments secure the block with one or more straps, or a hook-and-loop fastener such as Velcro.

In one aspect, an apparatus for applying adjustable pressure to a torso area of a patient comprises a wrap and a block. The wrap is configured to be worn by the patient. The wrap comprises a waist strap, a shoulder strap and a pouch. The waist strap is configured to extend around a torso of the patient, the shoulder strap is configured to extend over a shoulder of the patient and attach to the waist strap, and the pouch is configured to be positioned over a torso area of the patient. The pouch is configured to receive the block therein and to secure the block over the torso area of the patient to thereby apply a pressure by the block to the torso area of the patient, where the torso area of the patient is one among a plurality of torso areas of the patient over which the block may be secured within the pouch.

Various embodiments of the various aspects may be implemented. For example, the shoulder strap may be configured to be adjusted to adjust the pressure applied by the block to the torso area of the patient. The shoulder strap may comprise an elastic material and be configured to be adjusted by stretching the elastic material. The waist strap may be configured to be adjusted to adjust a compression applied by the wrap to the torso area of the patient. The waist strap may comprise first and second chest pads extending upwardly therefrom and configured to attach to the shoulder strap. The shoulder strap may be configured to be removably attached to the waist strap. The shoulder strap may further comprise on overstrap configured to extend over the pouch. The overstrap may be configured to be adjusted to adjust the pressure applied by the block to the torso area of the patient. The shoulder strap may comprise an elastic material and be configured to be adjusted by stretching the elastic material. The shoulder strap may comprise the pouch. The wrap may be reversible such that the block may be secured over a right torso area or an opposite left torso area of the patient. The pressure applied by the block may be high enough to reduce blood flow to the torso area to stop bleeding but still allow sufficient blood flow to the torso area to allow healing. The torso area of the patient may comprise a surgical incision site. The block may comprise an inelastic material. The pouch may be further configured to receive a filler block.

In another aspect, an apparatus for applying adjustable pressure to a torso area of a patient comprises a waist strap, a shoulder strap, a pouch and a non-compressible block. The shoulder strap is configured to attach to the waist strap, where the waist strap and shoulder strap when attached to each other are configured to surround a torso of the patient. The pouch is configured to be positioned over a torso area of the patient when the waist strap and shoulder strap are attached to each other and surrounding the torso of the patient. The pouch is configured to secure the non-compressible block over the torso area of the patient, and the shoulder strap is adjustable to adjust a pressure applied by the non-compressible block to the torso area of the patient.

Various embodiments of the various aspects may be implemented. The torso area of the patient may be one among a plurality of torso areas of the patient over which the non-compressible block may be secured within the pouch. The shoulder strap may further comprise on overstrap configured to extend over the pouch and to be adjusted to adjust the pressure applied by the block to the torso area of the patient. The shoulder strap may comprise the pouch. The pouch may be configured to allow for repositioning of the non-compressible block within the pouch by pressing on the outside of the pouch to move the block.

In accordance with another embodiment of the invention, a method of mitigating pocket bleeding and hematoma formation in a patient locates a localized torso area of the patient that may be subject to bleeding and/or hematoma formation. The localized torso area is at least in part covered by a bandage and/or dressing. Next, the method provides a pliable wrap having a non-inflatable solid block coupled with the wrap, and then secures the pliable wrap around the torso of the patient to align the solid block with the located localized torso area. The solid block is secured between at least a portion of the wrap and the bandage and/or dressing, at least in part covering the localized torso area. The wrap is secured to cause the wrap to apply a force, to the solid block, that produces a corresponding pressure on the localized torso area. The force applied to the solid block is high enough to cause the pressure to hinder blood flow to the localized torso area to promote clotting at the localized torso area. At the same time, the force applied to the solid block is low enough to cause the pressure to permit blood flow to the localized torso area to promote healing.

### BRIEF DESCRIPTION OF THE DRAWINGS

Those skilled in the art should more fully appreciate advantages of various embodiments from the following description discussed with reference to the drawings summarized immediately below.

FIG. 1 schematically illustrates a front view of the outer surface of a compression vest in a detached configuration in accordance with illustrative embodiments;

FIG. 2 schematically illustrates a rear view of the inner surface of a compression vest in a detached configuration in accordance with illustrative embodiments;

FIG. 3A schematically illustrates details of a fabric for a compression vest in accordance with illustrative embodiments;

FIG. 3B schematically illustrates a compression block urged towards a patient's body by a garment;

FIG. 3C schematically illustrates pressure gradient within a patient;

FIG. 4 schematically illustrates a front view in an attached configuration in accordance with illustrative embodiments;

FIG. 5 schematically shows a rear view in an attached configuration in accordance with illustrative embodiments;

FIGS. 6A-6B schematically illustrate an incision on the chest of a patient;

FIGS. 6C-6D schematically illustrate a front view in an attached configuration on a patient and a rear view in an attached configuration on a patient, respectively, in accordance with illustrative embodiments;

FIGS. 7A-7D schematically illustrate embodiments of compression blocks for use with a compression garment in accordance with illustrative embodiments;

FIGS. 7E-7F schematically illustrate cross sections of embodiments of compression blocks;

FIG. 7G schematically illustrates an embodiment of a compression block in a sheath;

FIG. 8 schematically illustrates an embodiment of a compression sleeve;

FIG. 9 is a flow chart illustrating a method of using a garment.

FIGS. 10A-10C illustrate details of a compression vest in a detached configuration in accordance with another embodiment.

FIGS. 11A-11F illustrate various views of a compression vest worn by a user in an attached configuration in accordance with another embodiment.

FIGS. 12A and 12B illustrate various views of a compression vest worn by a user in an attached configuration with an arm sling in accordance with another embodiment.

FIG. 13A and 13B illustrate arm straps of an arm sling in a detached configuration in accordance with another embodiment.

FIGS. 14A-14F illustrate various sequential views of a vest and arm sling showing a method of attaching the arm sling to the compression vest in accordance with another embodiment.

FIGS. 15A-15D illustrate various views of a block in accordance with another embodiment.

FIG. 16 illustrates a perspective view of a compression vest with a block in accordance with one embodiment.

FIGS. 17, 18, and 19 illustrate various example embodiments of a compression block having a donut, disc, and pillow shape, respectively, and that may be inflatable and/or have an opening therethrough.

### DETAILED DESCRIPTION

Illustrative embodiments mitigate pocket bleeding and hematoma formation in a patient by providing constant, adjustable pressure to a localized area of the patient's body. To that end, a mechanical, noninvasive garment has adjustable levels of compression to treat and/or substantially reduce the likelihood of hematomas from cardiac devices, such as pacemakers, defibrillators, loop recorders, and pressure monitoring systems placed underneath the skin in the human chest/torso. Among other configurations, the garment may be formed as a single unit with straps, and includes a compression block in a pouch of the garment. During use, the block applies specific pressure to a targeted area of the patient. The garment may be worn in a similar manner to a jacket, i.e., over the shoulder that is closest to the cardiac device. A first shoulder strap can be adjusted for appropriate contact, while a second strap (e.g., around the chest) may be adjusted to selectively apply pressure to the block, consequently applying pressure to the targeted area. Illustrative embodiments may be used over a device pocket (formed in a patient's body), which may have a hematoma, or may be used in a prophylactic fashion to avoid (or at least substantially reduce the likelihood that) a hematoma when they are expected.

During use, the patient may move around without being limited to a bed or chair. The garment also may enable the patient to carry on activities of daily living with minimal limitation. Accordingly, the garment may decrease the incidence of hematoma formation after cardiac device placement, especially when device implantation was done while the patient was on therapeutic anticoagulation. For patients on anticoagulants (e.g., heparin or enoxaparin), the garment may be used immediately after device implantation until the patient's anticoagulation levels settle to a prescribed therapeutic range. Also, the garment can compress a hematoma of the pocket that has already formed. As a result, the garment may reduce pain and suffering for the patient, decrease duration of hospitalization, decrease recurrent hospitalizations and additional interventions (e.g., reopening the device pocket and hematoma evacuations). Consequently, the garment potentially can reduce significant health care costs. Details of illustrative embodiments are discussed below.

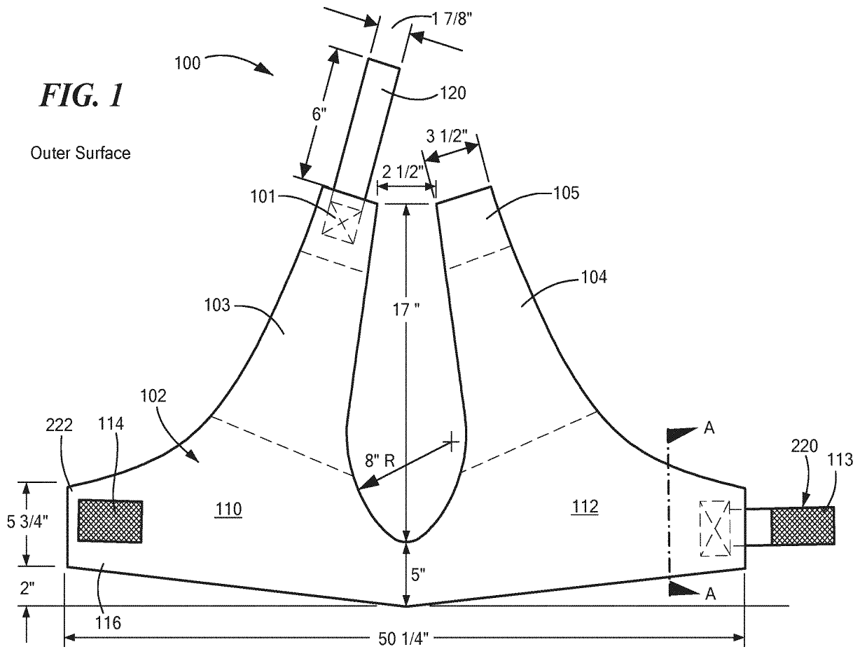

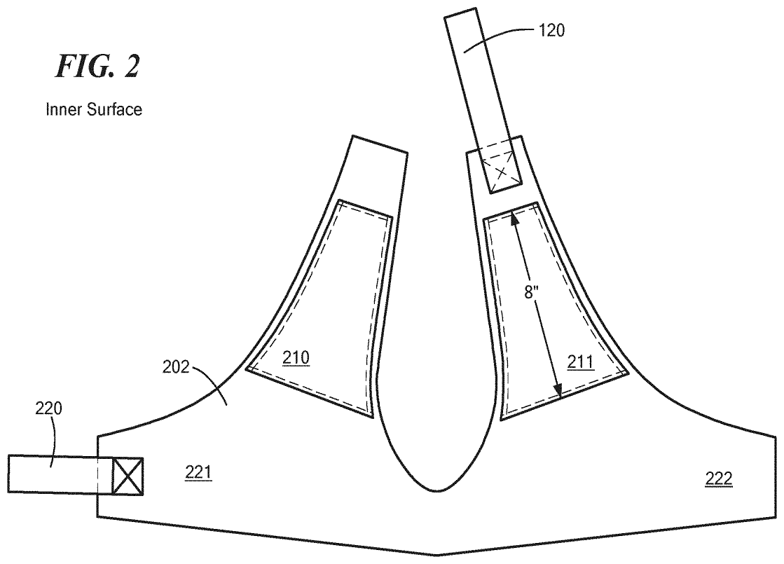

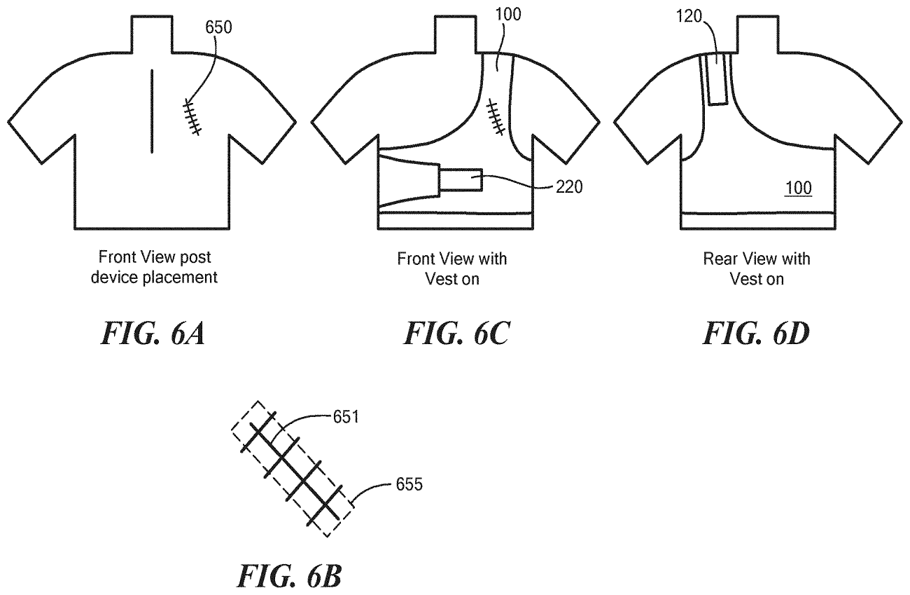

FIGS. 1 and 2 respectively show front and rear views of illustrative embodiments of a garment or vest 100 in a detached configuration. It should be noted that the specific dimensions in the drawing are exemplary only and thus, are not intended to limit various illustrative embodiments. FIGS. 6B and 6C schematically illustrate front and rear views, respectively, of a vest 100 worn by a patient.

The vest 100 is a single structure configured to anchor over the patient's shoulder and wrap around (e.g., form a loop around) the patient's upper chest in a circumferential fashion.

The vest 100 includes a waist band 110 configured to wrap around the patient's torso and secure the vest 100 to the patient. To that end, the waist band 110 includes a waist strap 220 coupled to a back flap 221. The waist strap 220 includes a hook and loop patch 113 (e.g., Velcro) configured to removably and adjustably couple to a counterpart hook and loop patch 114 on a front flap 222. The waist strap 220 may be useful in adjusting the top-to-bottom stretch and compression of the vest 100.

The vest 100 also includes left and right shoulder portions 101 and 105 extending from the waist band 110. In use, when worn by a patient, the left and right shoulder portions 101 and 105 are physically coupled by a shoulder strap 120, to secure the vest 100 around the patient's shoulder. For example, the strap 120 may go over the ipsilateral shoulder connecting the scapular portion 105 to the pectoral 101 portion of the vest.

Together, the shoulder strap 120 and waist strap 220 provide stretch and compression of the vest 100 in four directions (e.g., left and right across the patient's torso, and up and down relative to the patient's shoulders).

The vest 100 also includes at least one structure (e.g., 210; 211) for securing a compression block 700 (described below) to the vest 100. In illustrative embodiments, the structure 210; 211 may be a pouch, or sealable pouch, to hold the compression block 700. In some embodiments, the structure 210; 211 may include straps, belts, hook and loop fasteners, or other devices.

More specifically, a pouch 210 holds the compression block 700 adjacent to an area on a patient's body where pressure is desired. In illustrative embodiments, the pouch 210, 211 is on or in the inner surface 202 of the vest 100, so that the vest 100 presses the block 700 towards the patient.

Chest pads 103 and/or 104 hold a compression block 700 on the incision site (650) on patient's chest. In some embodiments, chest pad 103 and/or 104 of the vest 100 may have a surface that is larger than their respective pouches 210, 211. Moreover, the chest pads 103 and 104 provide a surface for applying a compressive force to the compression block 700 inside a pouch 210,211. The amount of compressive force applied by chest pads 103 and 104 to the compression block 700 may be adjusted using a shoulder strap 120 and/or a waist strap 220.

Some embodiments may come pre-attached at the shoulder strap and can be worn around the shoulder like a sling. Such embodiments may omit the waist band 110 and its strap 220.

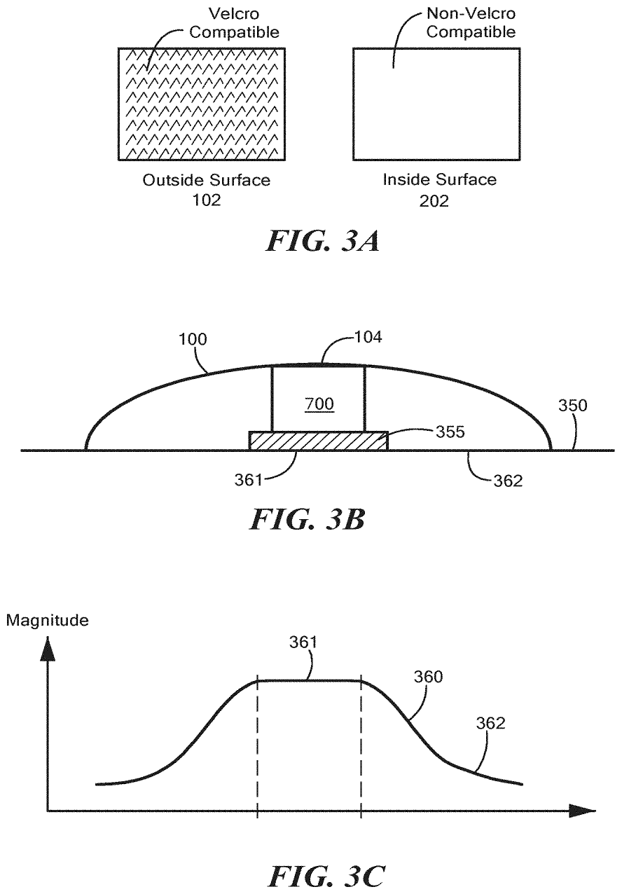

The vest 100 includes a stretchable body (e.g., waist strap 110 and/or shoulder portions 101 and 105) made up of stretchy or elastic materials, such as 2 mm thickness SBR Neoprene VELCRO compatible UBL finish, for example. In addition, as schematically illustrated in FIG. 3A, such materials may be waterproof and/or insulated. Moreover, the outer surface 102 of the vest may be VELCRO compatible and the inner surface 101 may be smooth and impervious to water.

FIG. 3B schematically illustrates a compression block 700 urged against a patient's body 350 by the stretchy material of a vest 100. In the embodiment of FIG. 3B, a bandage or dressing 355 is positioned between the compression block 700 and the patient 350. When worn by a patient, tension in the material of the vest 100 imparts a force to the compression block 700. The amount of the force may be adjusted by adjusting the straps 120 and/or 220. In addition, the amount of the force may be a function of the elastic properties and/or tightness of the material of the vest 100, in that the more the vest material is stretched, the greater the force it applies to the compression block 700. As discussed below, this force produces a pressure to the patient's body 350 (e.g., a portion of the patient's torso).

That force is high enough to cause the pressure to hinder blood flow to the localized torso area. Specifically, in illustrative embodiments, this pressure eventually aids in causing capillaries in the local area under pressure to coagulate, thus reducing and eventually stopping bleeding from the site. In addition, this pressure also preferably is low enough to mitigate the effect of a hematoma in that localized area. While a sufficiently high pressure is desired, such pressure also preferably is low enough to permit blood flow to the localized torso area. Accordingly, while reducing blood to the area to stop bleeding, blood still flows to the area to promote tissue health. As discussed below, a medical professional preferably can select the appropriate pressure to meet these competing demands.

The amount, location and gradient of the applied force may influence the efficacy of the vest 100. For example, as schematically by the compression gradient line 360 illustrated in FIG. 3C, the compression applied by the compression block 700 is high immediately beneath the compression block 700, but dissipates with distance from the block. The relatively high compression beneath the compression block 700 (e.g., at point at point 361), and relatively lower compression away from the block (e.g., at point 362) forces fluids within a hematoma or pocket outward from under the compression block 700. The structure of the compression block 700 directly impacts efficacy. Specifically, the inventors recognized that a solid compression block 700 produces a better-defined gradient than, for example, a bladder, which can be inflated. Accordingly, illustrative embodiments of the vest 100 and its compression block 700 should be beneficial to a patient with either or both a hematoma, and a high risk for pocket hematoma.

Specifically, various embodiments may be useful, for example, to:

1. Patients undergoing cardiac device implantation (pacemakers, cardioverter defibrillators, cardiac resynchronization therapy pacemakers & defibrillators, loop recorders, cardiac chamber pressure monitors) and chemotherapy ports who develop bleeding into the pocket and hematoma,

2. Patients who may be on antiplatelet and or anticoagulation therapy and may be at high risk for bleeding and hematoma formation,

3. Patients who may be on bridging therapy with heparin or enoxaparin immediately after cardiac device implantation for mechanical prosthetic valves, deep venous thrombosis, high risk atrial fibrillation patients, intracardiac thrombi etc.,

4. Patients who may be high risk for bleeding into pocket—elderly, renal failure, prolonged intervention, prior device extraction, pocket revisions, bleeding disorders etc., and/or

5. Patients who had a cardiac implantable electronic device (“CIED”) placed and developed a hematoma that needs that needs further care.

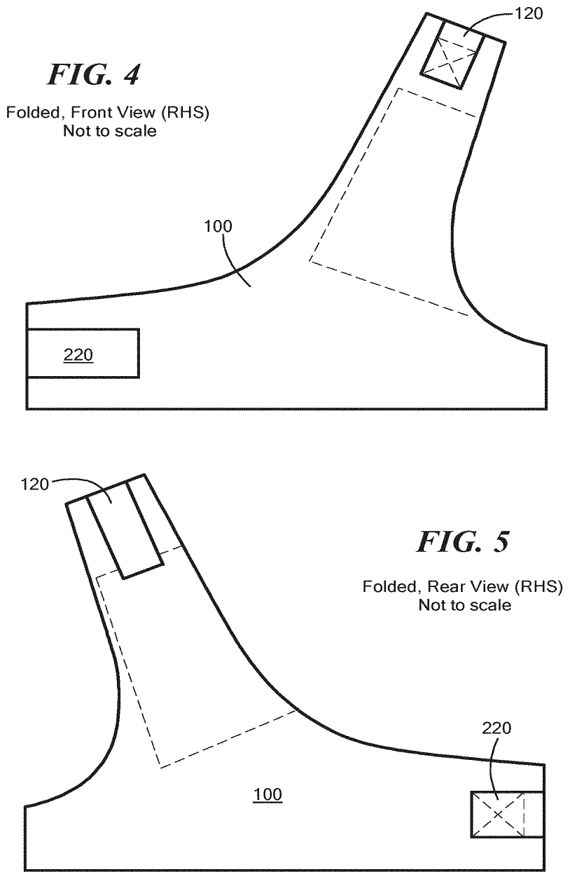

FIG. 4 and FIG. 5 show that the fabric pad may be folded approximately at its middle.

FIG. 6A, FIG. 6B and FIG. 6C schematically illustrate the patient with an incision 650 dressed on the left hand side of the body. FIG. 6B schematically illustrates a localized area 655 defined at least in part by the area where a compression block 700 meets a patient's body. For example, FIG. 6B schematically illustrates a localized area 655 near a patient's incision 650 and suture 651.

FIG. 6C schematically shows a front view in an attached configuration on the patient, while FIG. 6D shows a rear view of the vest 100 in an attached configuration on the patient. In some embodiments, from the front of the patient, the apparatus may be dressed on the patient and may approximately look like the front view illustrated in FIG. 6C. From the back of the patient, the dressed apparatus may approximately look like the rear view illustration in FIG. 6D. In other embodiments, the incision may be dressed on the right hand side of the body. In some embodiments, the adjustments described in reference to FIG. 1 may also be made when the vest is on the patient.

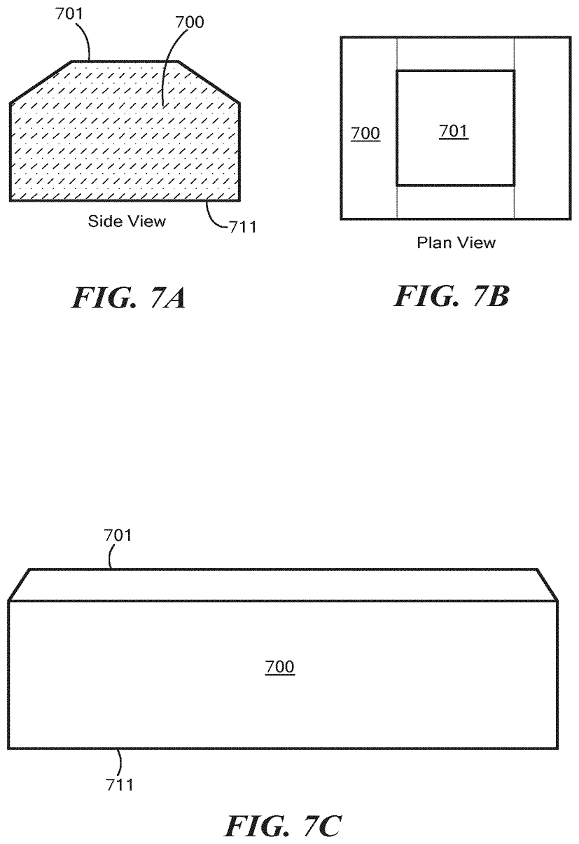

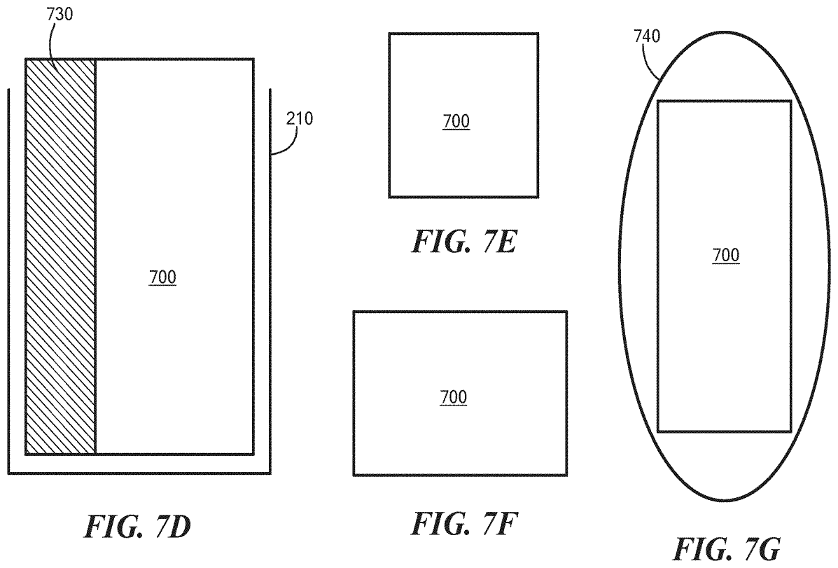

FIGS. 7A-7C schematically illustrate a compression block 700 in accordance with illustrative embodiments. The block 700 is solid, and in some embodiments is somewhat compressible. As a somewhat compressible member, the face of the block 700 preferably conforms to some extent with a facing surface of the portion of the patient to which it applies a pressure. The block 700 is not pneumatic, e.g., it is not an inflatable or deflatable bladder. Moreover, the block 700 is not formed from or include ice, which the inventors believe would be too hard and possibly injure the patient. In some embodiments, the compression block 700 may be made of gel foam, polystyrene foam (e.g., STYROFOAM), or other appropriate material can be placed to enhance the compression/pressure applied to the local area, as described further below.

The inventors have found that a solid compression block provides advantages over a bladder. For example, an inflatable bladder requires additional components, such as a pump mechanism to inflate the bladder, and a release mechanism to deflate the bladder. Such mechanisms increase the cost and complexity of the bladder, while decreasing the bladder's reliability and ease of use. In contrast, a solid block 700 is simple, durable, and reliable.

Moreover, the inventors believe that a bladder (whether inflatable or otherwise) deforms with an increase of internal pressure, and/or pressure against the patient, resulting a variable, and less precise pressure gradient and distribution or dispersal of pressure against the patient. Such an undesirable distribution of pressure can cause complications or impede the healing process. In contrast, pressure applied by a block 700 is more readily focused on a desired area, with less distortion and uncertainty.

As compared to a bladder, a solid block 700 may provide a more effective pressure suited to patient comfort.

For example, the location of pressure applied to a patient by a solid block 700 results in part from the size of the surface of the block that faces the patient. To that end, embodiments of block 700 may have surfaces of varying shapes and sizes.

In some embodiments, the block 700 has a square or rectangular cross section, as schematically illustrated in FIG. 7E and FIG. 7F, respectively, while in other embodiments, the block 700 has a trapezoidal cross section as schematically illustrated in FIG. 7A. For example, the top 701 of the block 700 may have the shape of the frustum of a pyramid. The height and length of the block 700 may be selected so as to cover the incision 650 and/or suture 651.

Illustrative embodiments of the block 700 have multiple surfaces, such as base surface 711, and narrower top surface 701. The localized area of pressure applied to the patient depends on which of those surfaces faces the patient. For example, a surface of the block 700 to be positioned facing the patent may be determined by a user based on the size of the incision 650, the suture 651, and/or the size of the desired localized area 655.

Further, in some embodiments, the pressure applied to a patient by a solid block 700 results in part from the interaction of the block 700 with pliable nature of the material of a garment 100. For example, pressure applied to the localized area may be determined at least in part by the elasticity of the garment, the position of straps, and the surface area of the block. In some embodiments, in which the material of the garment is elastic, the pressure delivered to the patent depends in part on the force applied by the fabric, and the compressibility of the block 700.

In some embodiments, the material selected for the compression block 700 may be foamed polystyrene. For example, the block may be an arteriotomy pressure point pad available from Steri-Systems Corp., P.O. Box 909, 1314 Fouth Avenue, Auburn, Ga. 30011 (Mfg. SKU: 100W; National Stock Number (NSN) 6515-01-464-2574). The density of the foamed polystyrene may vary. In preferred embodiments, material with 1 pound per cubic foot may be selected. However, preferred embodiments could vary density, such as from 0.5 to 1.5 pounds per cubic foot.

In some embodiments, the block 700 is non-compressible, or minimally compressible. If the block is too hard (e.g., steel; ice), the block 700 may fail to conform at all to the surface of the patient, and/or it may dig into the patient and distort the application of applied pressure.

To that end, in some embodiments, a minimally compressible block 700 may by a firm polystyrene material, or another material having a Young's modulus of between 2.0×10⁻¹⁰ dynes/cm² and 5.0×10⁻¹⁰ dynes/cm², and may be a polystyrene having a Young's modulus of 3.43×10⁻¹⁰ dynes/cm². In some embodiments, the block 700 may have a Young's modulus similar to that of polystyrene at 3 to 3.5 GPa. As another example, the block 700 of some embodiments may have a Young's modulus similar to that of Dow Chemical Company's Styrofoam Highload 60 product has a vertical compressive strength of 60 psi (pounds per square inch), meaning that the product experiences 10% deformation under 60 psi of pressure.

FIG. 7D schematically illustrates an embodiment that includes a filler block 730 configured to fit into a garment pouch, e.g., 210, alongside a compression block 700. When placed in a garment pouch, e.g., 210, a filler block 730 occupies space within the garment pouch 210 and forces the compression block 700 to one side or another of the garment pouch 210, thereby allowing adjustment and fine-tuning of the position of the compression block 700 relative to the garment 100, and relative to the localized area 655. In some embodiments, two or more filler blocks 730 may be used to position a compression block 700 within a pouch 210.

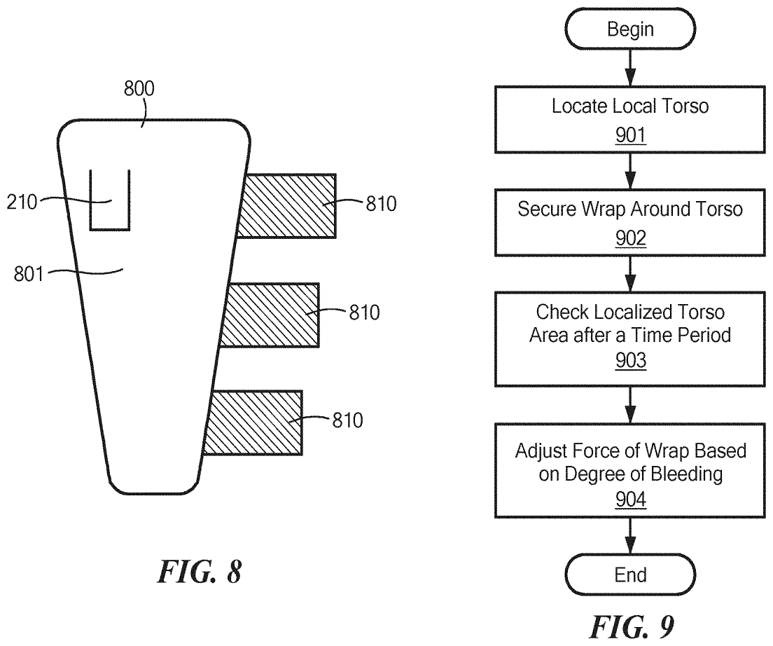

FIG. 8 schematically illustrates an alternative embodiment in which the garment 100 is in the form of a compression sleeve 800. The sleeve 800 of this embodiment is configured to wrap around a patient's limb, such as an arm or leg, and to be secured in position by one or more straps 810. The compression sleeve 800 includes a pouch 210 on an inner surface 801 for securing a compression block 700 between the inner surface 801 and the patient. In some embodiments, the sleeve 800 includes a stretchy material to apply pressure to the compression block 700, as described above in connection with the vest 100.

FIG. 9 is a flow chart illustrating a method of using a garment to selectively apply pressure to a localized area of a patient's body. The method may explain, for example, a method of mitigating pocket bleeding and hematoma formation in a patient.

The process begins at step 900, in which a medical professional or other user locates a localized area 655 of the patient's body (e.g., FIGS. 6A-6D). For example, the localized area 655 may be over a patient's incision 650, or a suture 651 closing an incision in the patient's torso. The localized torso area has a topography, and may be at least in part covered by a bandage and/or dressing.

Next, step 902 secures a pliable wrap (i.e., the garment 100) around the torso of the patient as discussed above. To that end, the garment 100 is secured so that the block is aligned with the part of the patient's torso requiring pressure. Among other things, as noted above, the patient may have undergone surgery to receive an implantable device. Accordingly, the garment 100 is oriented so that the block is over at least a part of the patient's torso needing the pressure. At this point in the process, the block is between the patient's body (e.g., the bandage and/or dressing 355) and the wrap.

The wrap thus applies a force to the solid block, which produces a corresponding pressure on the localized torso area. This force is due to the cooperation of the material forming the wrap and straps, as well as the material properties of the block. The medical professional ensures that the force is neither too high nor too low. Specifically, the force preferably is high enough to cause the pressure to hinder blood flow to the localized torso area. Accordingly, such a force should minimize or eventually stop bleeding at the local site. Importantly, however, the pressure also preferably is low enough to cause the pressure to permit blood flow to the localized torso area. In some embodiments, the force may be in a sufficient amount to cause the face of the block adjacent to the body to conform to some extent to the topography of at least a portion of the local region. This may further enhance the effect and controllability on hematomas and blood flow.

After the wrap has been secured for some prescribed amount of time (e.g., 30-90 minutes), a medical professional may check the progress of the localized torso area (step 903).

To that end, the medical professional may remove the wrap to view the localized torso area after the prescribed time period. If the medical professional recognizes fluid discharge from the localized torso area, then the wrap may not have been secured with a sufficient force. In that case, the process continues to step 904, in which the medical professional re-secures the wrap with a greater force than the force applied when initially secured. This greater force preferably is high enough to produce a greater block pressure to hinder blood flow to the localized torso area, but again, low enough to cause the pressure to permit blood flow to the localized torso area. Of course, when re-secured, the medical professional secures the wrap so that the block aligns with the local torso area being treated.

Alternatively, if in his/her professional judgement the local area is not bleeding/discharging fluid too much, then the medical professional will re-secure the wrap in the same manner (i.e., aligning the block with the local area) at approximately the same force.

Although the method illustrated by the flow chart in FIG. 9 is directed to a garment for use on a patient's torso, the method could also be applied to the use of garments on a patient's limb, such as sleeve 800 for example.

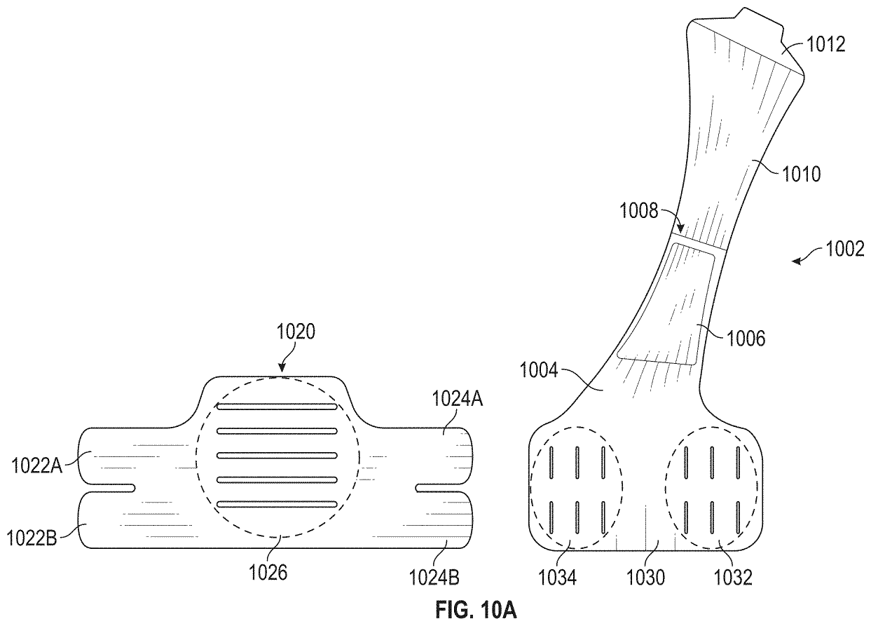

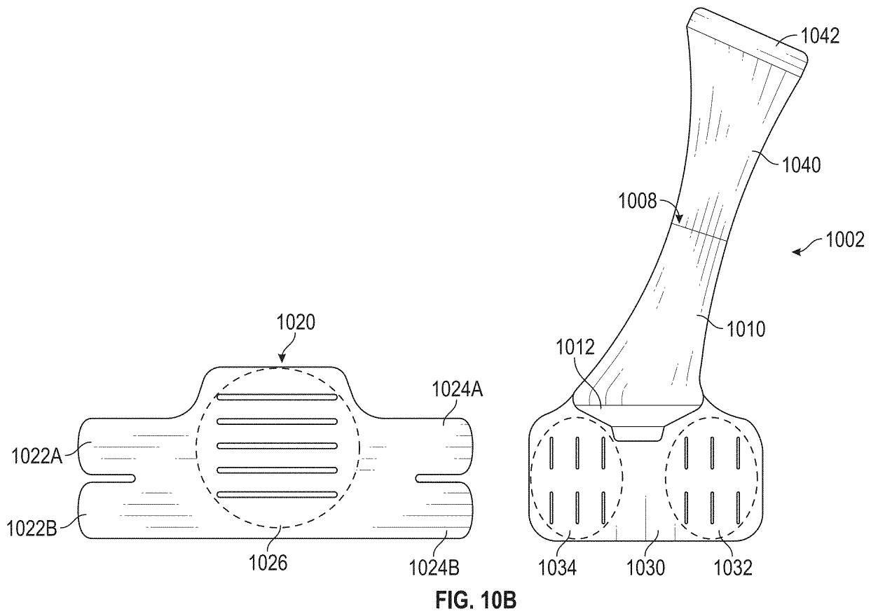

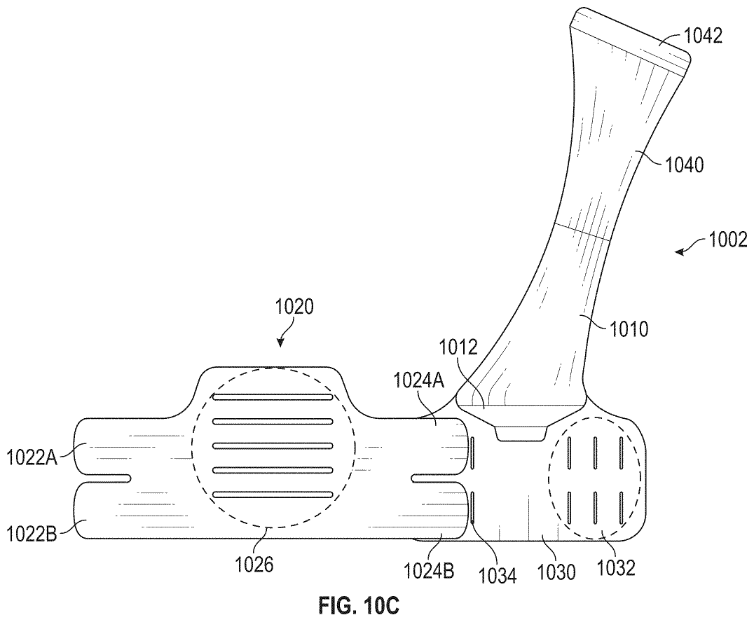

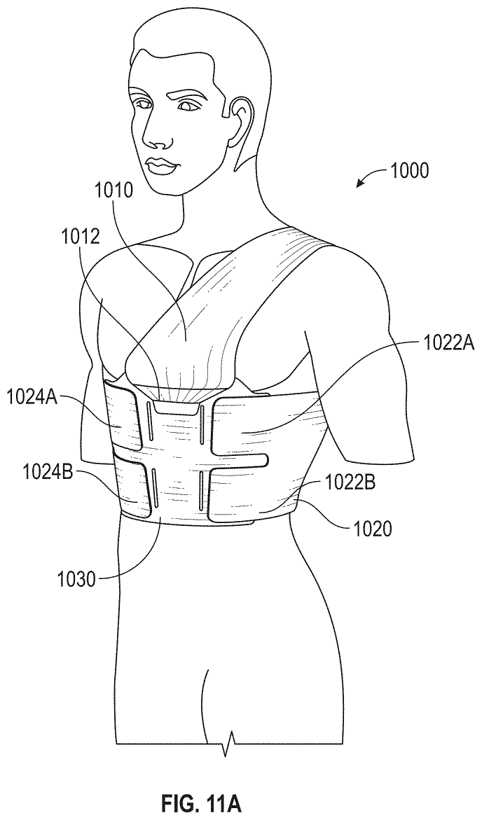

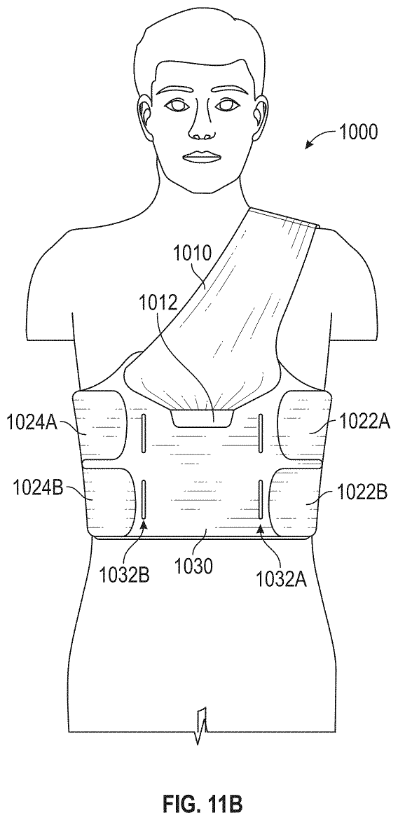

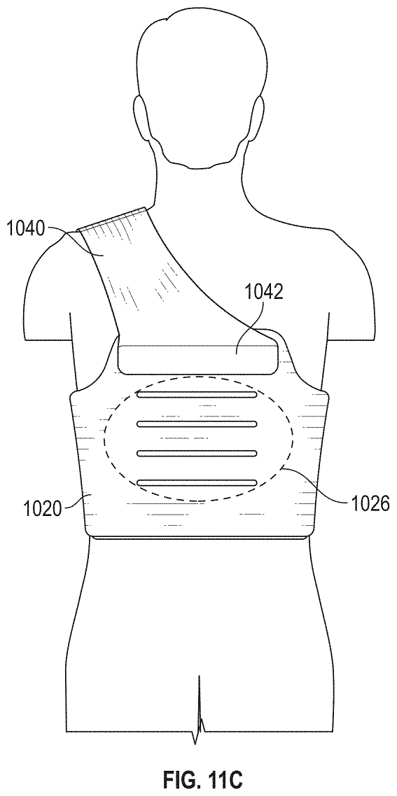

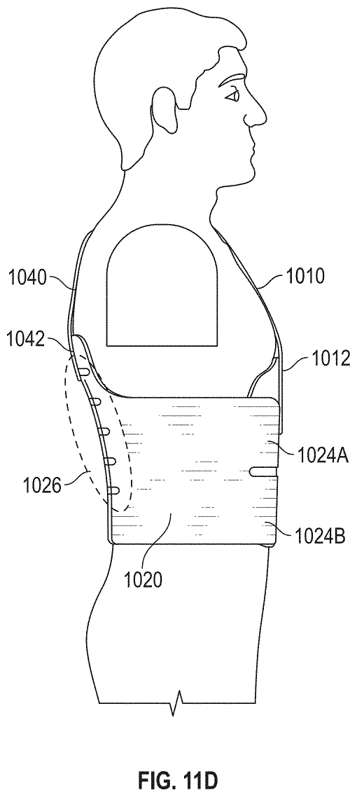

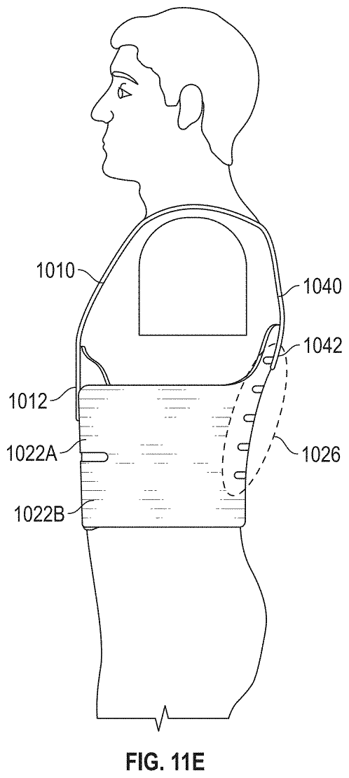

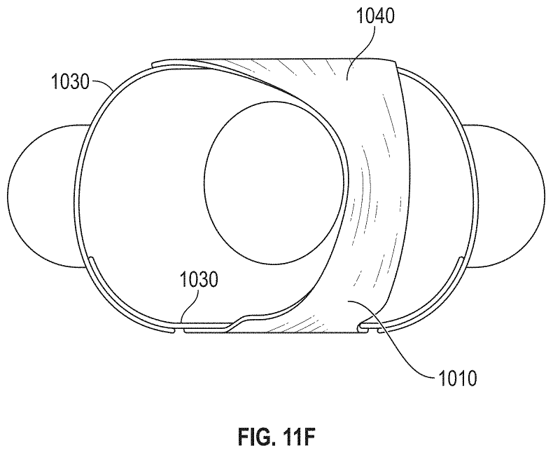

FIGS. 10A-10E schematically illustrate a detached configuration of a compression vest 1000 in accordance with illustrated embodiments. The compression vest 1000 can include a shoulder portion 1002 and a torso portion 1020. The shoulder portion 1002 can be draped over a user's shoulder as shown in, for example, FIGS. 11A-11F. The shoulder portion 1002 can removably connect to the torso portion 1020 to affix the compression vest 1000 to a user (for example, a patient).

With reference to FIGS. 10A-10E, the shoulder portion 1002 can include a coupling portion 1030, a first flap 1004, a second flap 1040, and a cover 1010. The coupling portion 1030, the first flap 1004, the second flap 1040, and the cover 1010 can together form a unitary piece. The first flap 1004 can extend at an angle from the coupling portion 1030. Such orientation of the first flap 1004 and the coupling portion 1030 can provide increased comfort for the user of the compression vest 1000. Additionally, such orientation of the first flap 1004 and the coupling portion 1030 can provide increased amount of compression force generated by the shoulder portion 1002.

The second flap 1040 can extend from the first flap 1004 such that the first flap 1004 is positioned between the coupling portion 1030 and the second flap 1040. As shown in FIG. 10A, the second flap 1040 can extend substantially in the same direction as the first flap 1004. The second flap 1040 can include a rear fastener 1042. The rear fastener 1042 can be positioned at a distal end (for example, an end opposite from the first flap 1004 and the coupling portion 1030, as shown in FIG. 10B) of the second flap 1040. The fastener 1042 can connect to a corresponding fastener, (that is, a fastener 1026) of the torso portion 1020.

The connection between the torso portion 1020 and the rear fastener 1042 of the second flap 1040 can generate compression force to prevent or reduce, for example, swelling and/or hematoma after cardiac procedures such as implant of subcutaneous cardiac devices.

In some embodiments, the width of the first flap 1004 and the second flap 1040 varies. The width of the first flap 1004 and the second flap 1040 can vary along the length of the first flap 1004 and the second flap 1040. For example, the width of a portion of the first flap 1004 approximate to the coupling portion 1030 can be greater than that of a portion of the first flap 1004 distal from the coupling portion 1030.

The cover 1010 can be attached to the shoulder portion 1002 between the first flap 1004 and the second flap 1040. As shown in FIG. 10B, the cover 1010 can be moveably coupled to the shoulder portion 1002 at a connecting point 1008. Optionally, the connecting point 1008 can be substantially positioned where the first flap 1004 and the second flap 1040 connects. The cover 1010 can include a fastener 1012 that can connect to a body of the coupling portion 1030, as shown in, for example, FIGS. 10B and 11B. The cover 1010 can have a first configuration in which the fastener 1012 is not connected to the coupling portion 1030 and a second configuration in which the fastener 1012 is connected to the coupling portion 1030.

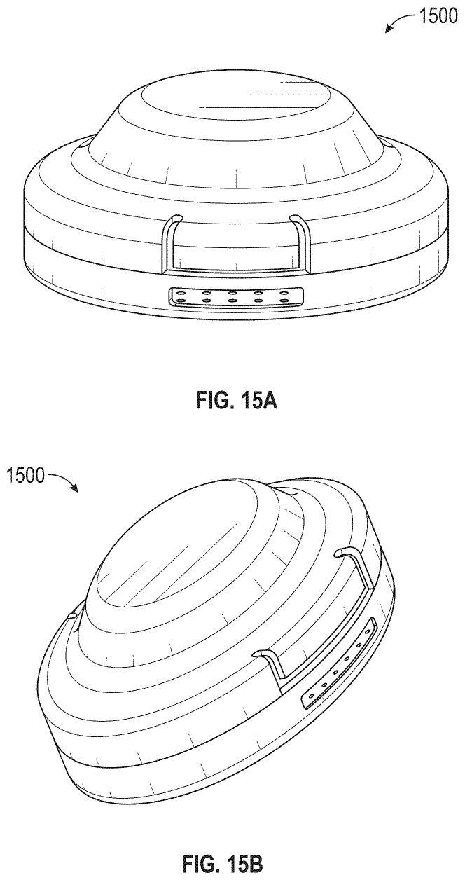

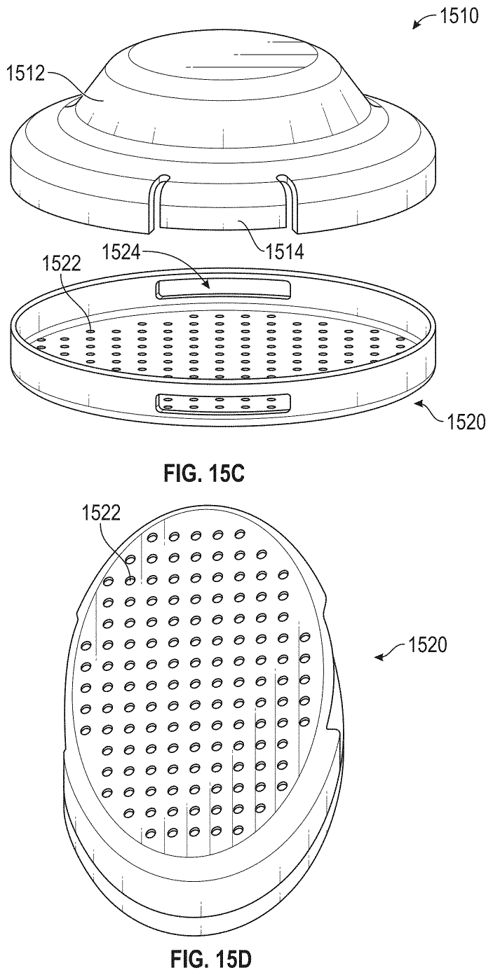

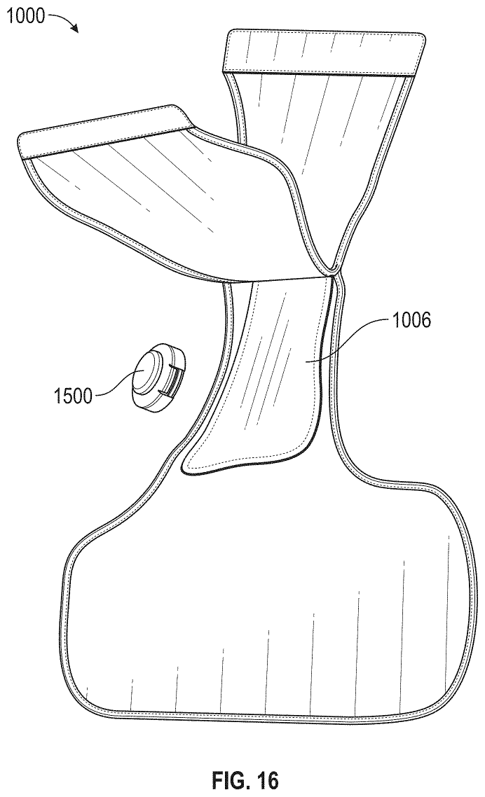

The shoulder portion 1002 can include a pouch 1006. The pouch 1006 can be coupled to the first flap 1004 and can receive a block 1500 (see, for example, FIGS. 15A and 16). When the cover 1010 is in the first configuration (that is, the fastener 1012 is not connected to the coupling portion 1030) the pouch 1006 can be accessible and the block 1500 can be placed inside the pouch 1006 or removed from the pouch 1006. When the cover 1010 is in the second configuration (that is, the fastener 1012 is connected to the coupling portion 1030), the pouch 1006 can be hidden from view and cover 1010 can prevent the block 1500 from being dislodged from the pouch 1006.

The coupling portion 1030 can include a first coupling portion 1032 and a second coupling portion 1034. For example, the second coupling portion 1034 can connect with corresponding fastener arms 1024A, 1024B of the torso portion 1020 as shown in FIG. 10C. The first coupling portion 1032 can connect with corresponding fastener arms 1022A, 1022B of the torso portion 1020.

The first coupling portion 1032 and the second coupling portion 1034 can each include visible markers that can indicate different attachment positions for the fastener arms 1022A and 1022B and the fastener arms 1024A and 1024B, respectively. By changing attachment positions of the fastener arms 1024A and 1024B (or the fastener arms 1022A and 1022B) on the second coupling portion 1034 (or the first coupling portion 1032), the compression vest 1000 can change in size and accommodate patients with different torso sizes.

FIGS. 11A-11F schematically illustrate various views of a user wearing the compression vest 1000. As shown in FIGS. 11A-11F, the torso portion 1020 can be wrapped around the torso of a user (for example, a patient) and the shoulder portion 1002 can extend about a shoulder of a patient and connect to the torso portion 1020. The torso portion 1020 can be wrapped around the torso of a patient such that a rear coupling portion 1026 of the torso portion 1020 faces outward (for example, facing in a direction away from the user). As such, the rear fastener 1042 of the second flap 1040 can connect with the rear coupling portion 1026. The shoulder portion 1002 can be positioned on the user such that the coupling portion 1030 and its first and second coupling portions 1032, 1034 can face outward (for example, facing in a direction away from the user).

As shown in FIGS. 11D and 11E, the rear coupling portion 1026 can include one or more visible markers that can indicate different attachment positions for the rear fastener 1042. By adjusting attachment position of the rear fastener 1042, the tension of the shoulder portion 1002 can be changed. For example, the tension of the shoulder portion 1002 may increase as the attachment position of the rear fastener 1042 is moved closer to a portion of the rear coupling portion 1026 closer to the bottom of the torso portion 1020 (for example, a portion of the torso portion farther from the shoulder of the patient), and vice versa.

Users with different heights and/or torso sizes can be accommodated by (1) adjusting the position of the rear fastener 1042 on the rear coupling portion 1026, and/or (2) adjusting the positions of the fastener arms 1022A, 1022B and 1024A, 1024B on the first and second coupling portions 1032, 1034 of the coupling portion 1030.

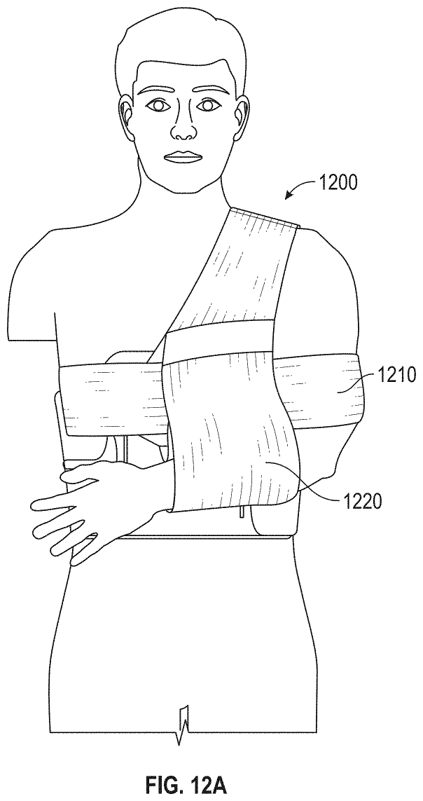

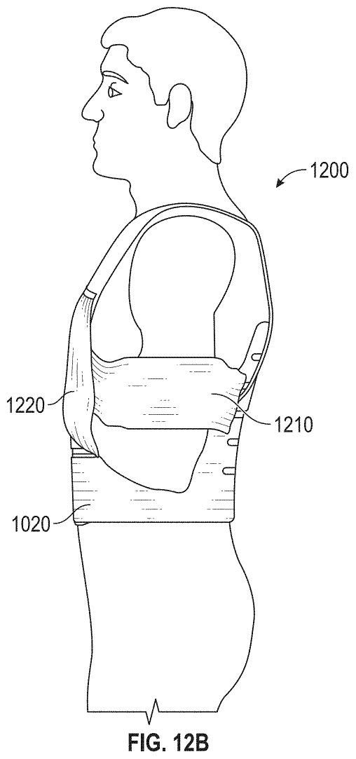

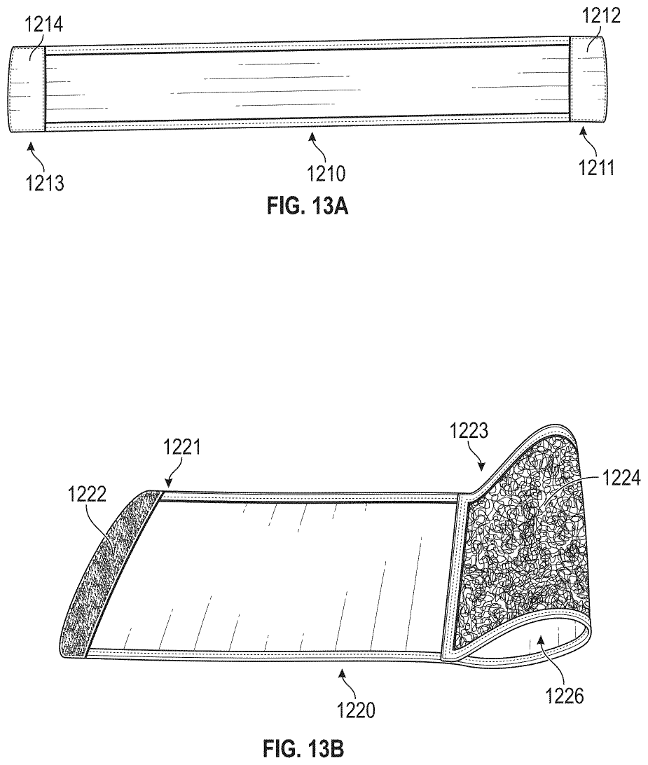

FIGS. 12A and 12B schematically illustrate the compression vest 1000 with an arm sling 1200 attached according to another embodiment. FIGS. 13A and 13B show arm sling 1200 in a detached configuration. The arm sling 1200 can include a first strap 1210 and a second strap 1220. The first strap 1210 can include a first end 1211, a second end 1213, and fasteners 1212, 1214. The fasteners 1212, 1214 can allow the first strap 1210 to be connected to the compression vest 1200. The fastener 1212 can be positioned proximate to the first end 1211. The fastener 1214 can be positioned proximate to the second end 1213.

The second strap 1220 can include a first end 1221, a second end 1223, and fasteners 1222, 1224, and an opening 1226. The fasteners 1222, 1224 can allow the second strap 1220 to be connected to the compression vest 1000 and/or the first strap 1210. The opening 1226 can be dimensioned to allow the first strap 1210 to extend through. The fastener 1222 can be positioned proximate to the first end 1221. The fastener 1224 can be positioned proximate to the second end 1223. The first end 1221 of the second strap 1220 can include the opening 1226. The fasteners may be removeable connections, such as Velcro, or other suitable mechanical attachments.

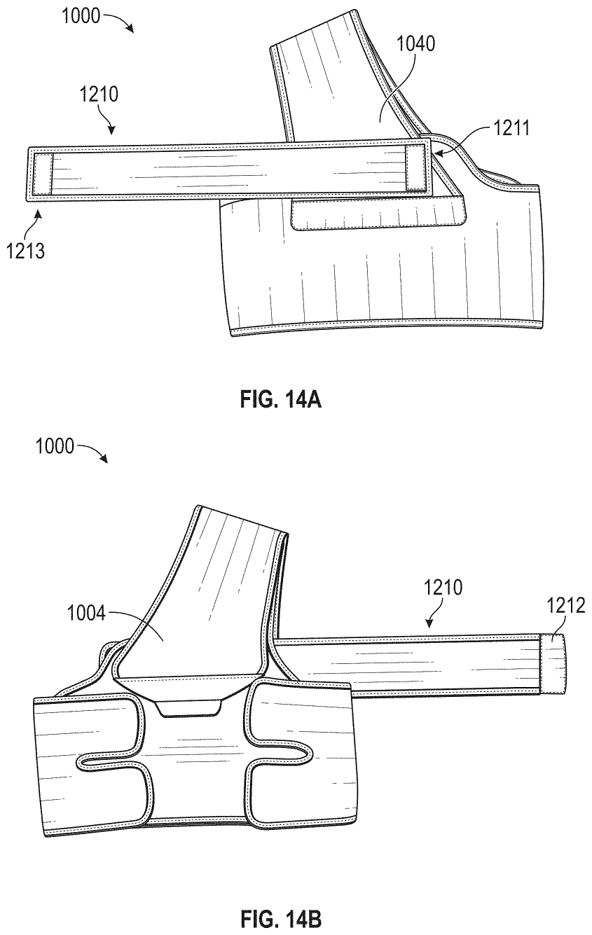

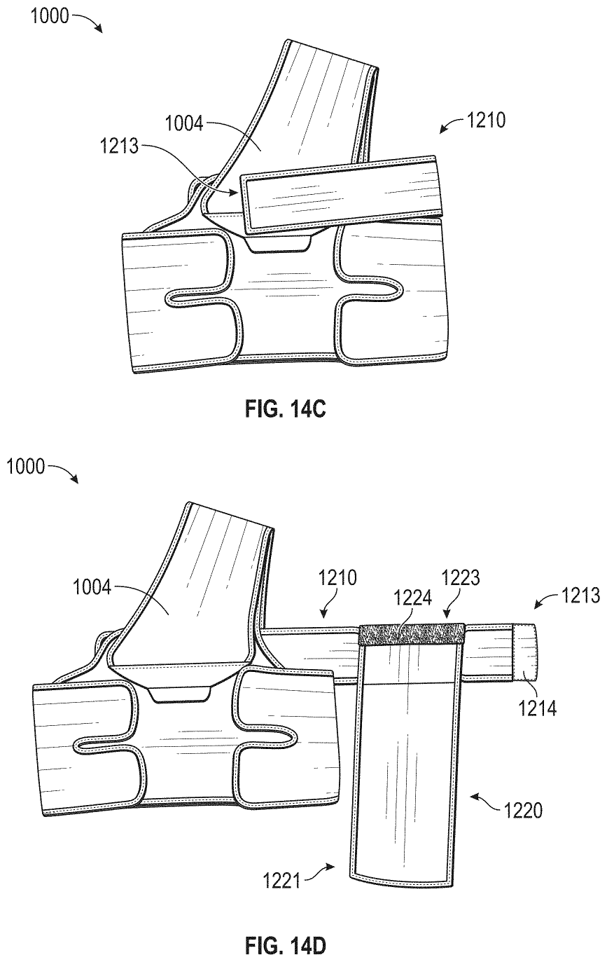

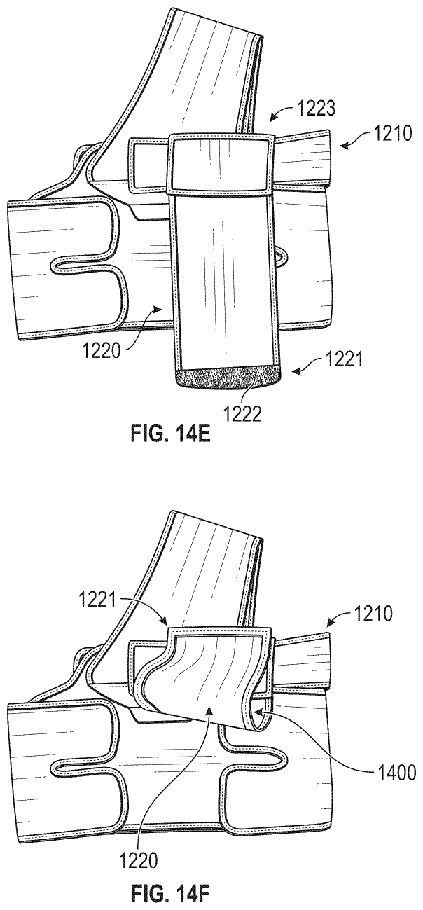

FIGS. 14A-14F illustrate an example method of attaching the arm sling 1200 to the compression vest 1000. As shown in FIG. 14A, one of the fasteners (for example, the fastener 1212 or the fastener 1214) of the first strap 1210 can be connected to the compression vest 1000. For example, the fastener 1212 can be connected to an outer surface of the second flap 1040. FIG. 14B illustrates a front view of the compression vest 1000 with the first strap 1210 attached to, for example, the second flap 1040. The second end 1213 of the first strap 1210 can wrap around a patient arm (for example, as shown in FIG. 12A) and the fastener 1214 can connect to the compression vest 1000. For example, the fastener 1214 can connect to the first flap 1004. When connected to the compression vest 1000, the first strap 1210 can restrict movement of a user's arm (for example, prevent the user from raising his/her arm).

The second strap 1220 can be used to support the user's forearm (for example, as shown in FIG. 12A). The opening 1226, as described herein, can receive the first strap 1210. FIG. 14D illustrates the first strap 1210 connected to, for example, the second flap 1040 of the compression vest 1000 and extending through the opening 1226 of the second strap 1220. The second strap 1220 can be positioned such that the fastener 1222 faces towards the compression vest 1000 when the first strap 1210 is wrapped around a user's arm. When the first strap 1210 is wrapped around a user's arm and is connected to, for example, the first flap 1004 of the compression vest 1000, the fastener 1222 of the second strap 1220 can additionally connect to, for example, the first flap 1004 of the compression vest 1000. This can help with securing the arm sling 1200 to the compression vest 1000.

Once the fastener 1224 is connected to, for example, the first flap 1004 of the compression vest 1000, the second end 1223 of the second strap 1220 can be wrapped around a forearm of the user (as shown in FIG. 12A) and allow the fastener 1224 to connect to either the first end 1221 of the second strap 1220 or other areas of the compression vest 1000 (for example, the first flap 1004). Once the second end 1223 is secured, for example, to the first end 1221, the second strap 1220 can form a loop 1400 that can hold the forearm of the user.

FIGS. 15A-15D schematically illustrate an example block 1500 according to another embodiment. With reference to FIGS. 15A-15D, the block 1500 can include a top portion 1510 and a bottom portion 1520. The top portion 1510 can include a protrusion 1512 and a connector 1514. The bottom portion 1520 can include openings 1522 and an opposing connector 1524. There may be a series of circular openings 1522 as shown. The openings 1522 may allow for thermal communication with the wound site. For example, a cold or hot pack may be placed inside the block 1500 to provide cooling or heating to the wound site. The protrusion 1512 may not be flexible and can be placed against, for example, a torso of a patient, to generate pressure. Optionally, the bottom surface of the bottom portion 1520 can be placed against, for example, a torso of a patient, to generate pressure. The connector 1514 and the opposing connector 1524 can interface to ensure that the top portion 1510 and the bottom portion 1520 remain connected. Various types of connecting/coupling/interlocking mechanisms or systems may be utilized to allow the top portion 1510 and the bottom portion 1520 to connect to each other. Optionally, the top portion 1510 and the bottom portion 1520 may be removably connected to each other.

FIG. 16 schematically illustrates a perspective view of the compression vest 1000 and the block 1500. As described herein, the block 1500 may be placed inside the pouch 1006. Optionally, more than one blocks 1500 may be placed inside the pouch 1006.

The block 1500 can optionally be in different shapes. For example, the block 1500 can be a donut-shaped (with an opening in the middle), a rectangular-shaped, pentagonal, hexagonal, or any other suitable shape. The donut-shaped block can generate pressure to, for example, a wound site, while preventing a point pressure (for example, a great amount of pressure applied in a small area). As such, donut-shaped blocks can generate pressure to areas surrounding, for example, a wound site without generating pressure to the wound site itself. The rectangular-shaped or hexagonal blocks can allow users (for example, care providers) to neatly arrange a number of blocks adjacent to each other without any gaps in between.

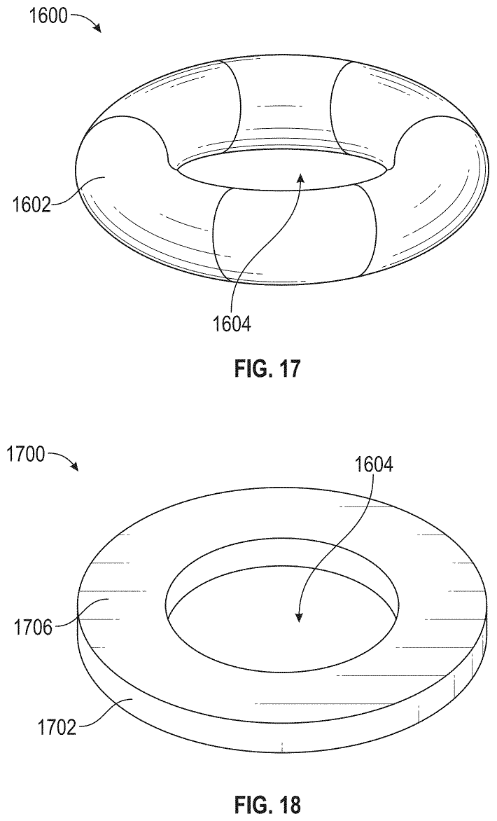

FIGS. 17, 18, and 19 illustrate various example embodiments of a compression block that can be used with any compression vest (for example, the compression vest 1000) described herein. As shown in FIG. 17, a compression block 1600 may be donut shaped and include a body 1602 including an opening 1604 defined centrally therethrough. The compression block 1600 can be positioned around a wound site such that the body 1602 can apply localized pressure in the areas surrounding the wound site while the opening 1604 can provide clearance and reduce pressure applied directly to the wound site. In the illustrated embodiment, the body 1602 has circular or oval cross-sections. Alternatively, the cross-sections of the body 1602 can be in other shapes such as square, rectangular, triangular, pentagonal, hexagonal, and so forth. In the illustrated embodiment, the opening 1604 is circular or oval in shape. Alternatively, the opening 1604 can be in other shapes such as square, rectangular, triangular, pentagonal, hexagonal, and so forth. The block 1600 may be inflatable, with a skin that collapses and inflated with air introduced via a valve on the block. The block 1600 may be solid, air-filled, or combinations thereof. In some embodiments the block 1600 may include multiple openings 1604, or it may not include an opening 1604. The outer profile of the block 1604 may have the rounded donut shape as shown, or it may be square, segmented, polygonal, rounded, combinations thereof, or other shapes.

FIG. 18 shows another embodiment of a compression block 1700 that may be used with any of the compression vests described herein. The compression block 1700 can include a body 1702 including an opening 1604 defined therethrough, which may be as described above. In some embodiments, in contrast to the body 1602, the body 1702 can include flat upper and/or lower opposite surfaces 1706 that can be positioned against the body of a patent. In the illustrated embodiment, the body 1702 has a rectangular cross-section. As described herein for the body 1602, so too the cross-sections of the body 1702 can be in other suitable shapes and may be inflatable, solid, etc. Further, any of the features of the block 1600 may be included in the block 1700, or vice versa.

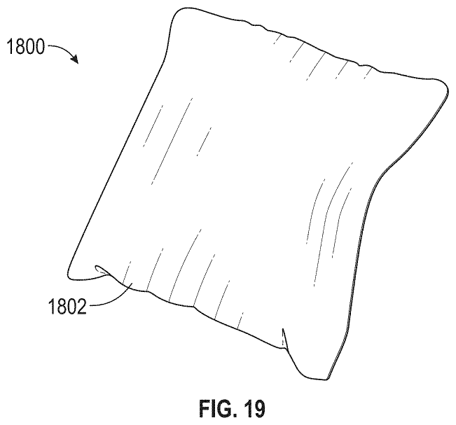

FIG. 19 shows another embodiment of a compression block 1800 that may be used with any compression vest described herein. The compression block 1800 can include a body 1802, which may be in a generally rectangular or square shape, such as a pillow shape. The four sides may be the same or different lengths from each other. There may be fewer or more than four sides. The body 1802 may store liquid-absorbent materials, temperature-controlling fluids and/or solids, medicines to promote healing, and so forth. As described herein, although the body 1802 is rectangular or square in shape in the illustrated embodiment, other suitable shapes (for example, hexagonal) may be used. The body 1802 may be inflatable, solid, etc. as described. Further, any of the features of the blocks 1600 or 1700 may be included in the block 1800, or vice versa.

Optionally, the blocks described herein (for example, the block 1500, donut-shaped block, rectangular-shaped block, and so forth) can be inflatable. By adjusting the amount of fluid (for example, water or air) inserted into the inflatable block, the amount of pressure generated and applied to, for example, a wound site can be varied. The fluid (for example, water) inside the inflatable device may be cooled (for example, frozen) or heated to provide cooling or heating during use.

Optionally, the blocks described herein (for example, the block 1500, donut-shaped block, rectangular-shaped block, and so forth) may be hollow or solid.

Optionally, the blocks described herein (for example, the block 1500, donut-shaped block, rectangular-shaped block, and so forth) may be rigid or flexible.

The following is a listing of reference numbers used herein:
100: Compression vest
101: Left shoulder portion, or scapular end, of vest
102: Outer surface of vest
103: Left chest pad portion of vest
104: Right chest pad portion of vest
105: Right shoulder portion, or pectoral portion, of vest
110: Waist strap or main body of vest
112: Right end of waist strap
113: Velcro fastener
114: Opposing Velcro fastener
116: Left end of waist strap
202: Inner surface of vest
210: Left pouch
211: Right pouch
220: Waist strap
221: Back flap
222: Front flap
350: Surface of patient's body
355: Bandage or dressing
360: Gradient graph
361: Pressure point
362: Lesser pressure point
650: Incision site
651: Suture
655: Localized area
700: Compression block
701: Top of compression block
711: Bottom of compression block
730: Filler block
740: Sheath
800: Sleeve
801: Inner surface of sleeve
810: Sleeve straps
1000: Compression vest
1002: Shoulder portion
1004: First flap
1006: Pouch
1010: Cover
1020: Torso portion
1022A, 1022B: Fastener arms
1024A, 1024B: Fastener arms
1026: Rear coupling portion
1030: Coupling portion
1032: First coupling portion
1034: Second coupling portion
1040: Second flap
1042: Rear fastener
1200: Arm sling
1210: First strap
1211: First end
1212: Fastener
1213: Second end
1214: Fastener
1220: Second strap
1221: First end
1222: Fastener
1223: Second end
1224: Fastener
1226: Opening
1400: Loop
1500: Block
1510: Top portion
1512: Protrusion
1514: Connector
1520: Bottom portion
1522: Opening
1524: Opposing connector
1600: Block
1602: Body
1604: Opening
1700: Block
1702: Body
1800: Block
1802: Body

Accordingly, unlike a tourniquet, when secured to the patient, the pressure that the garment applies is not sufficient to completely prevent blood flow. Rather, the garment applies a pressure sufficient to hinder but not completely prevent blood flow in the localized area 655 of the patient's body.

It is expressly understood that many other embodiments and modifications may be contemplated as included within the apparatus although not specifically illustrated. For example, it may be expressly contemplated that VELCRO fasteners may be placed in different positions or switched, namely a felt fastener exchange for hook fastener and vice versa. In a similar manner, although VELCRO may be preferred attachment means, any other appropriate attachment means, including bonded pressure adhesives, now known or later devised, may also be equivalently substituted. Still further, although it may be expressly contemplated that no strap, tape, or other means may be necessary to secure the compression block to the vest 100, the inclusion of such elements in some embodiments may be contemplated.

Complementary VELCRO fasteners can also be provided on the outside ends of the waist band 110 so that waist band 110 can be stretched and overlapped at its opposing ends and thus connected together.

Although the above discussion discloses various exemplary embodiments, it should be apparent that those skilled in the art can make various modifications that may achieve some of the advantages of the embodiments described herein without departing from the true scope of the inventive concepts. All such variations and modifications are intended to be within the scope of the present inventions as defined in any appended claims.

What is claimed is:

1. An apparatus for applying adjustable pressure to a torso area of a patient, the apparatus comprising:
a wrap configured to be worn by the patient, the wrap comprising a waist strap, a shoulder strap and a pouch, with the waist strap configured to extend around a torso of the patient, the shoulder strap configured to extend over a shoulder of the patient and attach to the waist strap, and the pouch configured to be positioned over a torso area of the patient; and
a block, wherein the pouch is configured to receive the block therein and to secure the block over the torso area of the patient to thereby apply a pressure by the block to the torso area of the patient, wherein the torso area of the patient is one among a plurality of torso areas of the patient over which the block may be secured within the pouch.

2. The apparatus of claim 1, wherein the shoulder strap is configured to be adjusted to adjust the pressure applied by the block to the torso area of the patient.

3. The apparatus of claim 2, wherein the shoulder strap comprises an elastic material and is configured to be adjusted by stretching the elastic material.

4. The apparatus of claim 1, wherein the waist strap is configured to be adjusted to adjust a compression applied by the wrap to the torso area of the patient.

5. The apparatus of claim 1, wherein the waist strap comprises first and second chest pads extending upwardly therefrom and configured to attach to the shoulder strap.

6. The apparatus of claim 1, wherein the shoulder strap is configured to be removably attached to the waist strap.

7. The apparatus of claim 1, wherein the shoulder strap further comprises on overstrap configured to extend over the pouch.

8. The apparatus of claim 8, wherein the overstrap is configured to be adjusted to adjust the pressure applied by the block to the torso area of the patient.

9. The apparatus of claim 9, wherein the shoulder strap comprises an elastic material and is configured to be adjusted by stretching the elastic material.

10. The apparatus of claim 1, wherein the shoulder strap comprises the pouch.

11. The apparatus of claim 1, wherein the wrap is reversible such that the block may be secured over a right torso area or an opposite left torso area of the patient.

12. The apparatus of claim 1, wherein the pressure applied by the block is high enough to reduce blood flow to the torso area to stop bleeding but still allow sufficient blood flow to the torso area to allow healing.

13. The apparatus of claim 1, wherein the torso area of the patient comprises a surgical incision site.

14. The apparatus of claim 1, wherein the block comprises an inelastic material.

15. The apparatus of claim 1, wherein the pouch is further configured to receive a filler block.

16. An apparatus for applying adjustable pressure to a torso area of a patient, the apparatus comprising:
a waist strap;
a shoulder strap configured to attach to the waist strap, wherein the waist strap and shoulder strap when attached to each other are configured to surround a torso of the patient;
a pouch configured to be positioned over a torso area of the patient when the waist strap and shoulder strap are attached to each other and surrounding the torso of the patient; and
a non-compressible block, wherein the pouch is configured to secure the non-compressible block over the torso area of the patient, and wherein the shoulder strap is adjustable to adjust a pressure applied by the non-compressible block to the torso area of the patient.

17. The apparatus of claim 16, wherein the torso area of the patient is one among a plurality of torso areas of the patient over which the non-compressible block may be secured within the pouch.

18. The apparatus of claim 16, wherein the shoulder strap further comprises on overstrap configured to extend over the pouch and to be adjusted to adjust the pressure applied by the block to the torso area of the patient.

19. The apparatus of claim 16, wherein the shoulder strap comprises the pouch.

20. The apparatus of claim 16, wherein the pouch is configured to allow for repositioning of the non-compressible block within the pouch by pressing on the outside of the pouch to move the block.
